(19) United States
(12) Patent Application Publication
(10) Pub. No.: US 2010/0226980 A1
(43) Pub. Date: Sep. 9, 2010

(54) NOVEL TABLET BASED ON S-ADENOSYL-METHIONINE

(75) Inventor: Marinette MOREAU, Saint Germain (FR)

Correspondence Address: BROWDY AND NEIMARK, P.L.L.C.;624 NINTH STREET, NW, SUITE 300, WASHINGTON, DC, 20001-5303, US

(73) Assignee: VETOQUINOL, Lure Cedex (FR)

(21) Appl. No.: 12/684,211

(22) Filed: Jan. 8, 2010

Related U.S. Application Data
(60) Provisional application No. 61/143,590, filed on Jan. 9, 2009.

(30) Foreign Application Priority Data
Jan. 9, 2009 (FR) 09/00058

(51) Int. Cl. A61K 9/44; A61K 31/7076; A61P 13/12

(52) U.S. Cl. 424/465; 424/467; 514/46

(57) ABSTRACT

A subject of the invention is a tablet for pharmaceutical use, particularly for veterinary use, particularly suitable for the chronic treatment of hepatic insufficiency, comprising at least one S-Adenosyl-Methionine salt. Said tablet has the advantage of being divisible and being capable of including an appetizing agent.

A subject of the invention is a tablet for pharmaceutical use, particularly for veterinary use. Said tablet is particularly well suited to the chronic treatment of hepatic insufficiency.

S-Adenosyl-Methionine (SAMe) salts are often indicated in the chronic treatment of hepatic insufficiency.

These are very hygroscopic compounds and thus difficult to handle outside of a controlled-hygrometry chamber. In fact, in the presence of moisture they become sodden with water and difficult to handle, taking on the texture of a gum.

The tablets of the prior art are coated, generally with an enteric coating, in order to avoid pyrosis and associated dyspepsia. This presents various problems.

When it is desired to cut up these scored tablets, the salts found at the cut surface are no longer coated and thus acquire the texture of a gum. Therefore, due to the enteric coating, the tablets are not suitable for cutting.

Moreover, due to the enteric coating, they cannot contain an appetizing agent.

It is therefore easily understood that there is a need for scored tablets comprising S-Adenosyl-Methionine salts, to which moreover it is possible to add adjuvants, for example one or more appetizing agent(s). In addition, due to the fact that these S-Adenosyl-Methionine salts cause pyrosis and dyspepsia, one of the other purposes of the invention is to provide scored tablets comprising S-Adenosyl-Methionine salts which moreover avoid pyrosis and dyspepsia.

Finally, yet another purpose of the invention is to provide a scored tablet comprising S-Adenosyl-Methionine salts, the active ingredient of which will be released in a pH-independent manner.

In order to resolve these various problems the inventors have developed a scored tablet the elementary particles of which are coated with at least one polymer which is neutral and insoluble in water, comprising S-Adenosyl-Methionine salts and at least one alkaline substance.

By elementary particles is meant each fine or extra-fine particle, grain or granule of the pure active ingredient, whether fixed or not fixed to a support.

Thus it is understood that according to the invention, the elementary particles of S-Adenosyl-Methionine salts and/or of an alkaline substance, are coated with at least one polymer which is neutral and insoluble in water, before being subjected to pressure in order to form a tablet.

Thus the final tablet is composed of particles (fine or extra-fine particle, grain or granule of the pure active ingredient) each coated with at least one polymer which is neutral and insoluble in water.

The invention is distinguished from the prior art by the absence of enteric coating and by the dissolution capacity of the tablet which is independent of the pH.

A first subject of the invention is thus a scored tablet comprising at least elementary particles of S-Adenosyl-Methionine salt, elementary particles of at least one alkaline substance, and at least one polymer which is neutral and insoluble in water, said elementary particles of said S-Adenosyl-Methionine salt and optionally those of the alkaline substance being individually coated with the polymer which is neutral and insoluble in water.

According to the invention, the S-Adenosyl-Methionine salts suitable for use can be chosen from all the salts known to a person skilled in the art, particularly S-Adenosyl-Methionine Paratoluene Sulphonate, S-Adenosyl-Methionine Paratoluene sulphate and S-Adenosyl-Methionine tosylate disulphate. Preferentially according to the invention S-Adenosyl-Methionine Paratoluene Sulphonate is used.

According to a particular embodiment of the invention, the S-Adenosyl-Methionine salt can be present in the tablet in a quantity of pure active ingredient comprised between 20% and 70%, preferentially between 25% and 55% of the total weight of the tablet. A person skilled in the art will have no difficulty in adapting the quantity of commercial S-Adenosyl-Methionine salts to be used, it being understood that this is a product which is never marketed pure.

Further, according to the invention, the alkaline substance makes it possible, on the one hand, to improve the stability of S-Adenosyl-Methionine and/or on the other hand to protect the stomach from the acid attack caused by the S-Adenosyl-Methionine salts.

By alkaline substance is meant according to the invention a compound which in solution has a pH strictly greater than 7.

Thus, when the tablet is administered, the gastric juice will be able to progressively dissolve the alkaline substance and the S-Adenosyl-Methionine simultaneously.

The diffusion of the S-Adenosyl-Methionine salts from the tablet can thus be accompanied by the diffusion of the dissolved alkaline substance, which can make it possible to reduce the aggressive acid effect of the S-Adenosyl-Methionine salts on the stomach.

According to the invention, the alkaline substance can advantageously be chosen from magnesium oxide, sodium bicarbonate, potassium bicarbonate or also aluminium hydroxide. Preferentially according to the invention, magnesium oxide will be used. Of course, the alkaline substance can comprise several of these compounds in a binary, ternary mixture, etc.

The quantity of alkaline substance which can be used per tablet can be determined in such a way as to comply with the “Acid neutralizing capacity” tests described in the American Pharmacopeia (US Pharmacopeia, page 1863, §301).

According to the invention, the elementary particles of the S-Adenosyl-Methionine salts and/or of the alkaline substance can have a minimum size of 50 microns. A person skilled in the art knows that in this regard there is no theoretical maximum size.

According to the invention, a polymer which is neutral and insoluble in water is a polymer which does not react chemically with the active ingredient and the solubility of which in water does not exceed 100 μg/ml (reference US Pharmacopoeia (United States Pharmacopeia (USP) #24 NF19) United States Pharmacopeia Editor; 25th edition (Jan. 1, 2000)).

The polymer which is neutral and insoluble in water can essentially be used as a barrier membrane. It can be chosen in order to control the release of the S-Adenosyl-Methionine salt throughout the length of the digestive tract, advantageously in a pH-independent manner.

The release of S-Adenosyl-Methionine will thus be a function of its diffusion through said neutral, water-insoluble polymer.

Moreover, it allows a highly effective masking of the taste of the S-Adenosyl-Methionine salts, embodying another advantage of the invention.

From the neutral, water-insoluble polymers suitable for use according to the invention, there can be mentioned celluloses such as methylcellulose, ethylcellulose, hydromellose, phthalate, cellulose acetate phthalate or methacrylates. According to the invention, ethylcellulose is preferably used.

According to a particular embodiment of the invention, the neutral, water-insoluble polymer can be present in the tablet in a quantity comprised between 25% and 50%, preferentially between 30% and 40% by weight with respect to the active ingredient.

According to the invention, the tablet can moreover comprise, simultaneously or individually,
an appetizing agent, such as for example a product of biological (powdered liver, meat, fish, etc.) or vegetable origin and preferentially a powder of vegetable origin, in a quantity comprised between 1% and 50%, preferentially between 3% and 30% with respect to the total weight of the tablet;
a neutral diluent/excipient such as for example lactose, mannitol, microcrystalline cellulose, starch and its derivatives, sugars and preferentially lactose or microcrystalline cellulose, in a quantity comprised between 10% and 70%, preferentially between 30% and 60% with respect to the total weight of the tablet;
a flow promoting agent, such as for example silica, stearate derivatives and any other agent known to a person skilled in the art which can be included in the composition of a tablet and preferentially silica, in a minimum quantity of 0.1% with respect to the total weight of the tablet;
a disintegration agent, such as for example derivatives of povidone or crospovidone, preferentially crospovidone, in a minimum quantity of 0.5% with respect to the total weight of the tablet;
   and any excipient which according to a person skilled in the art can be included in the composition of a tablet (e.g.: a lubricant).

A subject of the invention is also the production process for the tablet according to the invention.

According to a first variant of the process according to the invention, in a first stage a granulation is carried out of the S-Adenosyl-Methionine salt by spraying an aqueous solution of S-Adenosyl-Methionine salt in the presence of at least one neutral excipient, on which the elementary particles of S-Adenosyl-Methionine salt will be deposited and dried.

In a second stage, the granules obtained in the first stage, and optionally the elementary particles of the alkaline substance are coated with a solution comprising at least one polymer which is neutral and insoluble in water. The coating techniques which can be used in the second stage are those well known to a person skilled in the art and advantageously, the technique described in the experimental part in the paragraph headed “coating” of Example 1 will be used.

In a third stage, the tablets according to the invention are produced according to the standard techniques for tablet manufacture. In this stage it is possible to use only the coated granules obtained in the second stage, alone or in the presence of at least one other excipient usually used in pharmacy such as for example chosen from a phospholipid, a diluent, a flow promoting agent, an appetizing agent, a disintegration agent, a lubricant.

It is understood that according to this process, it is possible in the third stage for the tablets to comprise elementary particles of S-Adenosyl-Methionine salt coated with a neutral polymer and elementary particles of an alkaline substance coated with a neutral polymer or not coated.

According to the process of the invention, the granulation stage can be carried out by spraying a solution of S-Adenosyl-Methionine salt onto at least one neutral excipient in a fluidized bed, using the usual techniques in this field.

According to the invention, the neutral excipient can be for example microcrystalline cellulose, fine lactose crystals, mannitol, or also maltodextrin, preferentially microcrystalline cellulose, in a quantity comprised between 30% and 60% with respect to the total weight of the tablet.

In this variant, the quantity of salt can represent from 20% to 80%, preferably 50% to 70%, with respect to the total quantity represented by the quantity of salt added to the total quantity of neutral excipient, by weight.

According to a second variant of the process according to the invention, in a first stage, an aqueous solution of S-Adenosyl-Methionine salt is sprayed, for example in a hot chamber such that during the spraying, the solution dries almost instantaneously and only a powder of elementary particles of S-Adenosyl-Methionine salt remains, in a second stage, the elementary particles of S-Adenosyl-Methionine salt obtained in the first stage are coated as previously with a solution comprising at least one neutral, water-insoluble polymer and in a third stage the tablets according to the invention are produced using the usual techniques for tablet manufacture.

In this stage it is possible to use only the coated granules obtained in the second stage, alone or in the presence of at least one other excipient from those usually used in pharmacy such as for example chosen from a phospholipid, a diluent, a flow promoting agent, an appetizing agent, a disintegration agent, a lubricant.

Advantageously according to the invention, the tablets will comprise, as well as the coated granules obtained in the second stage, at least an alkaline substance as defined previously.

According to the invention, the salt solution used in the first stage of the process is a commercial salt solution prepared at a concentration which can be comprised between 10 and 60% weight/weight, preferentially 20 to 50%.

According to the process of the invention, the quantity of coating can represent from 10 to 20% by weight with respect to the total weight of the granule obtained in stage 1 plus the coating weight.

The tablet obtained after using one of the processes according to the invention is particularly well suited to the chronic treatment of hepatic insufficiency. It has a reduced acidity in order to avoid causing gastric hyperacidity and is therefore not aggressive for the stomach. It can moreover comprise an appetizing agent and it is capable of being cut.

It has the advantage moreover of releasing the active ingredient in a manner which is independent of the pH.

A subject of the invention is also the use of a tablet according to the invention as a medicament.

A subject of the invention is also the use of a tablet according to the invention for the preparation of a medicament intended for the treatment or the prevention of chronic renal insufficiency.

Finally, a subject of the invention is a tablet according to the invention comprising moreover at least one extract of milk thistle (Silybum marianum) seeds, known as silymarin, advantageously at least one flavolignan extracted from milk thistle seeds, very advantageously natural or synthetic Sylibin (silibinin). According to this option, as the extract of milk thistle seeds is only optional, it is thus not regarded as an active ingredient; it can advantageously be added to the coating only.

The invention can be used in mammals, in particular human beings, preferably in animals, more particularly in small animals such as dogs and cats.

The following examples illustrate the present application without however limiting it.

### EXAMPLE 1

### Preparation of Tablets

The preparation of a coated tablet containing SAM-e Paratoluene Sulphonate is carried out in two stages: a granulation stage followed by a coating stage.

1. Stage of Adsorption of SAM-e Paratoluene Sulphonate on an Inert Support

An aqueous solution of SAM-e Paratoluene Sulphonate (26 to 50% m/m) is sprayed in a fluidized bed onto a mix of neutral excipients comprising microcrystalline cellulose and fine lactose crystals according to the proportions given in the table below.

| Components | Proportions/% |
|---|---|
| SAMe paratoluene sulphonate * | 60.00 |
| Fine lactose crystals | 10.00 |
| Microcrystalline cellulose PH 102 | 30.00 |
| TOTAL | 100.00 |

* the SAM-e Paratoluene Sulphonate is in aqueous solution at approximately 50%

2. Coating

The powder obtained previously is then coated. For this stage, neutral, water-insoluble polymers were used, such as cellulose derivatives such as ethylcellulose. For this stage, the spraying process using a GLATT-type fluidized bed (well known in the prior art) will be used, at a temperature compatible with the neutral polymer used for the coating, for a variable period of time according to the quantities and size of the batches to be coated.

The three formulae are prepared according to the proportions given in the table below (in %).

| Components | Formula N°1 | Formula N°2 | Formula N°3* |
|---|---|---|---|
| Internal phase | 85.00 | 90.00 | 81.00 |
| Ethylcellulose | 15.00 | 10.00 | 19.00 |
| Total | 100.00 | 100.00 | 100.00 |

*Formulae No.s 1 and 2 were produced with a single coating, unlike Formula N° 3 which underwent two successive coatings (the number of coatings is according to the SAM-e content and palatability results).

3. Final Mixture

The table below gives several typical formulae for tablets according to the invention:

| | Composition | Composition | Composition |
|---|---|---|---|
| Components | SAM-e N°1 | SAM-e N°2 | SAM-e N°3 |
| Coated SAM-e** particle | 67.44 | 76.65 | 67.44 |
| Silybin/phospholipids | 0.00 | 0.00 | 4.76 |
| Diluent (lactose, | 17.81 | 8.60 | 13.05 |
| microcrystalline cellulose, | | | |
| etc.) | | | |
| Flow promoting agent | 1.00 | 1.00 | 1.00 |
| (silica, etc.) | | | |
| Alkalizing agent | 1.25 | 1.25 | 1.25 |
| (Magnesium oxide, etc.) | | | |
| Appetizing agent | 5.00 | 5.00 | 5.00 |
| Disintegration agent | 7.00 | 7.00 | 7.00 |
| (Acdisol, crospovidone, | | | |
| etc.) | | | |
| Lubricant (PRUV, etc.) | 0.50 | 0.50 | 0.50 |
| Total | 100.00 | 100.00 | 100.00 |

**obtained in stage 2

4. Pharmacokinetic Tests

### EXAMPLE 2

### Dissolution Tests of the Tablets According to the Invention

Two dissolution tests of tablets according to the invention having 100 or 400 mg of S-Adenosyl-Methionine salt [SAME] were carried out with Apparatus 2 of the European Pharmacopoeia. The SAME tablets are controlled-release tablets.

### Equipment

| Description | Reference |
|---|---|
| Spindle dissolution apparatus | VARIAN, VK7025, or equivalent |
| UV spectrophotometer | VARIAN, Cary 50 or equivalent |
| 1-mm quartz cells | HELLMA |

### Chemical Reagents

| Substances | Quality | Risks |
|---|---|---|
| Water | Ultra-pure, Milli-Q type | — |
| Hydrochloric acid R (HCl) | For analysis | Corrosive |
| Potassium dihydrogen phosphate | For analysis | — |
| (KH₂PO₄) | | |
| Sodium hydroxide (NaOH) | For analysis | Corrosive |
| SAMe paratoluene sulphonate | Reference substance | — |

### Preparation of the Dissolution Medium

### 0.1 M HCl Medium

85 ml of 37% HCl was introduced into a 10-litre beaker and was made up to full volume with water. The pH was checked and adjusted (1.0±0.1) as required.

### KH₂PO₄ Buffer Medium pH 7.2

68.3 g of KH₂PO₄ and 13.5 g of NaOH were introduced into a 10-litre beaker. Water was added to make it up to 10.0 L and the medium was magnetically stirred until dissolution. Checking and adjustment (7.20±0.05) was carried out as required

### Preparation of the Control

Exactly 213.7 mg of SAMe paratoluene sulphonate was weighed and placed in a 1000.0 ml phial, and HCl 0.1 M medium was added to make it up to 1000.0 ml.

Stirring was carried out using ultrasound until dissolution.
Note: No difference in absorbency was noted between a control prepared in 0.1 M HCl and a control prepared in KH₂PO₄ buffer pH 7.2.

### Dissolution Parameters

| | |
|---|---|
| Volume of dissolution medium per bowl: | 900 ml |
| Stirring speed of the blades: | 100 rpm |
| Temperature of the bath: | 37.0 ± 0.5° C. |
| Optical path: | 1 mm |
| Detection wavelength: | 257 nm |

### Conduct of the Dissolution

The tablets were allowed to dissolve for 1 hour in HCl medium. The dissolution was then continued in KH2PO4 buffer medium pH 7.2 for 2 hours.

Samples were then taken at 5, 10, 15, 20, 30, 45, 60, 70, 80, 90, 105, 120, 135, 150, 165 and 180 minutes (the samples from 5 to 60 minutes were taken in an acid medium and the others in the buffer medium at pH 7.2).

### Estimation of the Dissolution of the SAMe

The percentage dissolution of the SAMe was then estimated as a function of the absorbency of the control solution.

The percentage dissolution of the SAMe was then estimated by measuring the absorbance of the residual SAMe at the wavelength of 257 nm.

The measurement parameters were

| | |
|---|---|
| Volume of dissolution medium per bowl: | 900 ml |
| Stirring speed of the spindles: | 100 rpm |
| Temperature of the bath: | 37.0 ± 0.5° C. |
| Optical path: | 1 mm |
| Detection wavelength: | 257 nm |

Results:

The tests show that whatever the concentration of SAMe in the tablet, the dissolution profile and kinetics are independent of the pH of the dissolution medium.

### OTHER EXAMPLES

In order to confirm the results obtained in vitro, pharmacokinetic tests were carried out on dogs. The results showed that the SAMe is absorbed in the stomach as soon as it is administered and that it is not degraded. The pharmacokinetic profiles of the different dogs are homogeneous and vary very little.

In order to verify the effectiveness of the taste masking, palatability tests were carried out on dogs and cats. The results showed a significant improvement in palatability.

1. Scored tablet comprising at least elementary particles of S-Adenosyl-Methionine salt, elementary particles of at least one alkaline substance, and at least one polymer which is neutral and insoluble in water, said elementary particles of S-Adenosyl-Methionine salt and optionally those of the alkaline substance being individually coated with the neutral, water-insoluble polymer.

2. Tablet according to claim 1, characterized in that the S-Adenosyl-Methionine salt is chosen from S-Adenosyl-Methionine Paratoluene Sulphonate, S-Adenosyl-Methionine sulphate and S-Adenosyl-Methionine tosylate disulphate, preferentially S-Adenosyl-Methionine Paratoluene Sulphonate.

3. Tablet according to claim 1, characterized in that the S-Adenosyl-Methionine salt present in the tablet is in a quantity of pure active ingredient comprised between 20% and 70%, preferentially between 25% and 55% of the total weight of the tablet.

4. Tablet according to claim 1, characterized in that the neutral, water-insoluble polymer is chosen from celluloses, such as methylcellulose, ethylcellulose, hydromellose, phthalate, cellulose acetate phthalate or methacrylates, preferentially ethylcellulose.

5. Tablet according to claim 1, characterized in that the neutral, water-insoluble polymer is present in the tablet in a quantity comprised between 25% and 50%, preferentially between 30% and 40% by weight with respect to the active ingredient.

6. Tablet according to claim 1, characterized in that the alkaline substance is chosen from magnesium oxide, sodium bicarbonate, potassium bicarbonate or also aluminium hydroxide, preferably magnesium oxide.

7. Tablet according to claim 1, characterized in that it also comprises at least one extract of milk thistle (Silybum marianum) seeds, advantageously at least one flavolignan extracted from of milk thistle seeds, very advantageously natural or synthetic silybin (silibinin).

8. Tablet according to claim 1, characterized in that it comprises moreover, simultaneously or individually,
an appetizing agent, such as for example a product of biological (powdered liver, meat, fish, etc.) or vegetable origin and preferentially a powder of vegetable origin, in a quantity comprised between 1% and 50%, preferentially between 3% and 30% with respect to the total weight of the tablet;
a neutral diluent/excipient such as for example lactose, mannitol, microcrystalline cellulose, starch and its derivatives, sugars and preferentially lactose or microcrystalline cellulose, in a quantity comprised between 20% and 70%, preferentially between 30% and 60% with respect to the total weight of the tablet;
a flow promoting agent, such as for example silica, stearate derivatives and any other agent known to a person skilled in the art which can be included in the composition of a tablet and preferentially silica, in a minimum quantity of 0.1% with respect to the total weight of the tablet;
a disintegration agent, such as for example povidone derivatives and preferentially crospovidone, in a minimum quantity of 0.5% with respect to the total weight of the tablet;
and any excipient which according to a person skilled in the art can be included in the composition of a tablet (e.g.: a lubricant).

9. Process for the production of a tablet according to claim 1, characterized in that in a first stage the S-Adenosyl-Methionine salt is granulated by spraying an aqueous solution of S-Adenosyl-Methionine salt in the presence of at least one neutral excipient; in a second stage the granules obtained in the first stage are coated with a solution comprising at least one neutral, water-insoluble polymer; in a third stage the tablets according to the invention are produced.

10. Process according to claim 9, characterized in that the neutral excipient is microcrystalline cellulose, fine lactose crystals, mannitol, or also maltodextrin, preferentially microcrystalline cellulose, in a quantity comprised between 30% and 60% with respect to the total weight of the tablet.

11. Process according to claim 9, characterized in that the quantity of salt can represent from 20% to 80%, preferably 50% to 70%, with respect to the total quantity represented by the quantity of salt added to the total quantity of neutral excipient, by weight.

12. Process for the production of a tablet according to claim 1, characterized in that in a first stage an aqueous solution of S-Adenosyl-Methionine salt is sprayed in order to obtain a powder of elementary particles of S-Adenosyl-Methionine salt; in a second stage said elementary particles of S-Adenosyl-Methionine salt obtained in the first stage are coated with a solution comprising at least one neutral, water-insoluble polymer and in a third stage the tablets according to the invention are produced.

13. Process according to claim 9, characterized in that in the third stage, the tablets comprise coated granules obtained in the second stage, alone or in the presence of at least one excipient from those usually used in pharmacy such as for example chosen from a phospholipid, a diluent, a flow promoting agent, an appetizing agent, a disintegration agent, a lubricant.

14. Process according to claim 9, characterized in that the salt solution used in the first stage is an aqueous salt solution, the concentration of which is comprised between 10 and 60% weight/weight, preferentially from 20 to 50%.

15. Process according to claim 9, characterized in that the quantity of coating represents from 10 to 20% by weight with respect to the total weight of the granule obtained in stage 1 plus the coating weight.

16. A method of using a tablet according to claim 1 as a medicament.

17. A method of using a tablet according to claim 1 for the preparation of a medicament intended for the treatment or the prevention of chronic renal insufficiency.
